(19) United States
(12) Patent Application Publication
(10) Pub. No.: US 2019/0380564 A1
(43) Pub. Date: Dec. 19, 2019

(54) ENDOSCOPIC CAP WITH REINFORCED WALL

(71) Applicant: Assistance Publique - Hôpitaux de Pahs (AP-HP), Paris (FR)

(72) Inventor: Jérôme VIALA, ECOUEN (FR)

(21) Appl. No.: 16/307,858

(22) PCT Filed: Jun. 6, 2017
(86) PCT No.: PCT/FR2017/051421

§ 371 Date: Aug. 21, 2019

(30) Foreign Application Priority Data
Jun. 6, 2016 (FR) 1655163

(51) Int. Cl. A61B 1/00

(52) U.S. Cl. CPC A61B 1/00137; A61B 1/00089; A61B 1/0014

(57) ABSTRACT

An endoscope cap and methods for manufacturing and using the cap, the cap including (1)—a sheath (2) with a proximal opening and a distal opening (4) facing the proximal opening and having a shape flaring towards its distal opening, —an attachment (3) suitable for attaching the cap to the distal end of an endoscope, with the proximal opening facing or surrounding the distal end of the endoscope, the wall of the sheath being made of a structure having sufficient strength to withstand perforation and/or incision by a sharp and/or pointed object.

### RELATED APPLICATIONS

The present application is a National Phase entry of PCT Application No. PCT/FR2017/051421, filed Jun. 6, 2017, which claims priority to FR 1655163, filed Jun. 6, 2016, the contents of each being incorporated herein by reference in their entireties.

### TECHNICAL FIELD

This invention relates to the field of endoscope accessories for the extraction of foreign bodies from a human or animal organism.

This invention relates particularly to an endoscopic cap designed to be fixed onto the distal end of an endoscope in order to cover the object that is to be extracted.

References between square brackets ([..]) refer to the list of references at the end of the description.

### PRIOR ART

The ingestion of foreign bodies covers situations that are very different in children and in adults. A child between 1 and 3 years will explore his or her external environment through the most innervated parts of his or her body, namely fingers and mouth. It is natural for the child to ingest a wide variety of objects. In 2007, American Poison Control Centers had listed 125 000 cases of the ingestion of foreign bodies [1]. Some of these objects were dangerous due to their slicing or cutting nature [2]. Accidental ingestion by teenagers and adults are less frequent and deliberate ingestions with the objective of autolysis are becoming more frequent. Ingested objects are then frequently and deliberately traumatising [3]. Patients suffering from behavioural disorders due to encephalopathy or an autistic syndrome, can also ingest traumatising objects.

These slicing, perforating or cutting objects can cause serious lesions in the digestive tract, up to perforation [4]. These perforations are frequently complicated by nearby infections, mediastinitis or peritonitis. If they pass through the digestive wall, these traumatising foreign bodies can cause injury to other organs such as the lungs, liver, spleen or can trigger serious haemorrhages by lesions of large vessels. The oesophagus is the most frequently injured organ because of its small diameter, its contractility and the thinness of its wall.

Several international recommendations require endoscopic extraction of these traumatising foreign bodies as quickly as possible [2], [5]. It they go beyond the ligament of Treitz, they would become inaccessible to digestive endoscopy and could traumatise the small intestine. The risk of perforation has been estimated at 4.8% in a retrospective adult study [6]. However, endoscopic extraction is not risk-free because the return passage of the foreign body through the oesophagus can cause new oesophageal lesions due to pulling on the endoscope.

One known solution to limit the risk of oesophageal lesion during withdrawal, is to use an endoscope overtube. The overtube consists of tunnelling the oesophagus with a plastic tube with an inside diameter that accepts the passage of the endoscope [3]. However, its diameter makes it unusable for young children.

Another solution is to use an endoscope protection cap. This type of cap is made of flexible plastic and is fixed to the end of the endoscope using an elastic [7]. However, this material is not very resistant to perforation or laceration. Furthermore, the fact that it is folded in front of the end of the endoscope severely restricts the field of vision, making it difficult to grip the foreign body. It can even camouflage the fact that the foreign body has perforated the cap. In this case, extraction can injure the oesophagus without the endoscopist even being aware of it, thus being conducive to an even more extensive lesion.

Note that this type of difficulty is not specific to humans, it can also occur in animals that have swallowed a perforating or cutting foreign body, either accidentally or as a result of an action by a badly intentioned person.

Furthermore, such difficulties can occur whenever the objective is to remove a foreign body from a cavity in an organism other than the stomach without injuring tissues while removing the endoscope.

Therefore the problem that embodiments of this invention aims to solve is to reduce the risk of injury to tissues when withdrawing an endoscope after the endoscope has gripped the foreign body, when withdrawing a cutting or perforating foreign body inside the cavity of a human or animal organism.

### DETAILED DESCRIPTION

This problem is solved by the subject of this invention.

Therefore the purpose of embodiments of this invention is an endoscopic cap comprising:
a protection envelope formed by a wall folded on itself such that the envelope comprises a proximal opening and a distal opening facing the proximal opening, and the wall having a shape tapered outwards from its proximal opening to its distal opening, in a basic configuration,
an attachment means arranged so that the cap can be attached on the distal end of an endoscope with the proximal opening facing or around the distal end of the endoscope,

the wall of the envelope being made of a structure with sufficient strength to resist perforation and/or incision by a pointed and/or cutting object.

Thus, the inventors chose to keep the use of a cap but to work on the structure of its walls. They realised that they could thus obtain good results while protecting the tissues during the withdrawing of the endoscope.

The cap can thus be fixed to the end of the endoscope that is introduced into the organism, end called the distal end of the endoscope.

The attachment means enables either positioning of the proximal opening facing the distal end of the endoscope, or enables the endoscope to pass through this proximal opening. Therefore vision using the endoscope beyond this proximal opening is possible.

Therefore the distal opening of the cap that is opposite the proximal opening in the cap is the part furthest from the endoscope. Therefore, in facing the proximal opening, this enables vision using the endoscope beyond this distal opening, even in the basic configuration, thus facilitating the search for the foreign body.

The basic configuration is the configuration that will be used when the endoscope returns outside the organism. The tapered shape of the cap in this basic configuration is such that it surrounds the foreign body and thus protects the tissues during this return path.

The cap according to embodiments of the invention can optionally include one or several of the following characteristics:
the envelope can be in the form of a bell, the proximal opening being arranged at the apex of this bell, and the distal opening being arranged at the base of this bell; throughout this application, the term “bell” refers to any shape tapered outwards from the proximal opening to the distal opening, which includes particularly symmetric shapes of revolution with a curved generating line or conical shapes; this bell shape facilitates the display, the approach, gripping and surrounding of the foreign body;
in its basic configuration, the envelope defines a housing and the wall of the envelope may include a stiffener arranged to surround this housing; this stiffener enables simple manufacturing to prevent any risk of perforation or cutting of the cap by the foreign body; furthermore, during the search for the foreign body, this stiffener helps to hold the distal opening more firmly open at its maximum diameter, without folding, and facing the proximal opening and thus facilitating vision;
the stiffener may be formed from one or several wires; this is a simple way of fabricating a stiffener;
the stiffener, and particularly the wire(s), may be formed from a metallic or carbon material; these materials have good resistance to incision, while remaining flexible to a certain extent, thus also improving the passage of the cap into the organism and surrounding the foreign body;
the stiffener may be formed from at least one element chosen from among:
one or more metal wires,
one or more wires used to make tubular endoprostheses,
a set of fibres, particularly carbon fibres; these materials can be used to make a stiffener with very good resistance to incision;
the stiffener may be formed from one or more wires composed of a shape memory material, particularly a nickel and titanium alloy and particularly nitinol; this can confer a shape on the cap by deformation of its basic configuration to facilitate insertion into the organism; once in the cavity, the cap is exposed to the warmth of the organism and resumes its basic configuration;
the wire(s) can form a mesh; this improves the resistance of the cap to incision; for example, this mesh can be composed of meshes with a mesh size of about 1 mm² side dimension;
the envelope can include at least one layer composed of at least one external covering polymer covering the stiffener on the exterior of the housing; this protects tissues while the endoscope is being inserted and/or withdrawn;
the envelope may include another layer composed of at least one internal covering polymer covering the stiffener inside the housing; this reinforces the resistance of the cap to perforation; furthermore, when the cap is designed to fold back on itself so be inserted into the organism, this also protects the tissues;
the internal cover polymer and the external cover polymer may be identical;
the wall of the envelope may be formed from one polymer only or several polymers; in the latter case, it does not necessarily have a stiffener; they are polymers with sufficient resistance to be able to resist perforation and/or incision by a pointed and/or sharp object; these polymers may in particular be reinforced by fillers;
the attachment means may comprise a retention sleeve with a first end intended to be fixed on the distal end of the endoscope, the retention sleeve having a second end fixed on the envelope such that the sleeve opens up in the proximal opening; this sleeve forms a simple means of fastening the cap, while making it possible to position the proximal opening to enable vision beyond the opening;
the sleeve and the wall of the envelope can be made in a single piece of the same material; in particular, they can be obtained being mold together or molding the sleeve together with a reinforcing cover layer;
the sleeve may be composed of a material composed of one or several polymers, called sleeve polymers;
the material forming the sleeve may be elastic and/or porous; this makes it easier to slide it onto the distal end of the endoscope and increase its adhesion to the endoscope;
the sleeve may have an internal surface comprising at least one recess and/or at least one relief; this improves adhesion of the sleeve to the distal end of the endoscope, particularly when the endoscope comprises a shape complementary to this recess or this relief;
the wall may be flexible so that the envelope can be put into a folded configuration, in which the envelope is folded back on itself around the attachment means, and to change from this configuration to an unfolded configuration, corresponding to the basic configuration and in which the envelope is turned back in the other direction such that it is positioned adjacent to the attachment means; once the cap is in place on the endoscope, the endoscope can be turned over to open up the field of vision in front of the endoscope even more;
according to the preceding paragraph and when the envelope comprises a stiffener, the stiffener may begin only at the top of the envelope while remaining exclusively in the envelope, the attachment means not having a stiffener, the zone adjacent to the stiffener thus being more flexible than the wall of the envelope thus forming a hinge around which the envelope can be turned back on itself in its folded configuration;
the envelope may be arranged so as to pass, by deformation, from a compacted configuration to an expanded configuration corresponding to the basic configuration, in its compacted configuration the envelope having a smaller taper than the basic configuration or not having a taper at all, while keeping the distal opening facing the proximal opening; this makes it easier to insert the cap without turning it over, while maintaining good visibility in front of the endoscope,
according to embodiments of the invention, in the compacted configuration, the envelope can have folding portions folded on themselves, with folds extending longitudinally from the distal opening to the proximal opening, and in which the folding portions are unfolded in the expanded configuration;
the cap can include a stiffener made of a shape memory material, the austenitic phase transformation end temperature is between 34 and 42 degrees, the envelope being arranged so as to have the deployed configuration in the austenitic phase of this material; this makes it easy to make the cap, facilitates its deformation so that it can be inserted into the organism, and deployment of the cap, which takes place without any action by the endoscopist;
the elasticity of the wall of the envelope can be such that in the compacted configuration, this wall is under a stress generating a return force tending to bring the wall from its compacted configuration to its expanded configuration, the cap comprising one or more retaining elements holding the envelope in the compacted configuration; the envelope can thus be extended by the action of this or these retaining elements by an actuator of the endoscope; this or these retaining elements may be one or more wires encircling the envelope in the compacted configuration;
the wall forming the envelope can be discontinuous and split over its entire length, from a proximal end to a distal end, such that the envelope can be obtained by rolling the wall on itself along the direction of its length, so as to close the envelope between the proximal end that will be around the endoscope, and the distal end that thus forms the edges of the distal opening; the result is thus a device that can be easily adapted to various endoscopes;
the envelope can thus have dimensions such that its housing contains a volume about 4 centimeters (cm) diameter and 5 to 6 cm long; the distal opening can have a diameter of at least 4 cm.

Another purpose of embodiments of the invention is an endoscope equipped with a cap according to embodiments of the invention, this cap being fixed on the distal end of the endoscope by the attachment means of the cap with the proximal opening of the cap facing or around the distal end of the endoscope. Thus equipped, the endoscope can thus include an elastic surrounding the cap and the distal end of the endoscope while being clamped so as to retain or to complete retention of the cap on the distal end.

Another purpose of embodiments of the invention is a method of fabrication of an endoscopic cap according to embodiments of the invention, the cap comprising an envelope comprising a proximal opening and a distal opening facing the proximal opening and having a shape of a basic configuration, this shape tapering outwards from its proximal opening to its distal opening, the fabrication method comprising:
a step for fabrication and/or conformation of a stiffener either in the form of a handle for which the shape corresponds at least partly to the shape of the basic configuration, namely in the form of a cap,
a step to cover the stiffener by a layer composed of at least one covering polymer on at least one of the stiffener faces,

the stiffener material and/or the covering polymer(s) being resistant to perforation and/or incision by a pointed and/or cutting object, said manufacturing method.

As a result the stiffener can memorize the shape adapted in the basic configuration. Thus, the cap that will include this stiffener can be easily deformed before it is introduced into the organism and it will expand once it is inside the cavity.

According to the fabrication method according to embodiments of the invention, the stiffener material can be chosen from among shape memory materials for which the temperature at the end of the change to the austenitic phase is between 34 and 42 degrees. The stiffener conformation step is performed before the covering step is implemented at a sufficiently high temperature so that this shape will become the shape memorized by the stiffener, this shape being the shape that the stiffener will have in the basic configuration of the cap, the fabrication method also comprising:
a step in which the stiffener is cooled below the martensitic phase transformation end temperature of the stiffener material,
a step in which the stiffener is compacted by reducing the taper of the envelope by deformation of the stiffener while keeping the distal opening facing the proximal opening, this compaction step being done below the martensitic phase transformation end temperature of the stiffener material and before or after the covering step.

The cap is thus ready for insertion before being used for extraction.

The covering step can then be done:
by gluing and/or welding a wall made of polymer material(s) on the stiffener,
by over molding of one or several polymer materials on or around the stiffener,
by dipping in a polymerisable material, so that the material is polymerised around the stiffener.

Another purpose of embodiments of the invention is use of the cap according to embodiments of the invention in a method of extraction of a foreign body from an internal cavity in a human or animal organism, comprising:
a step to pick up an endoscope fitted with a cap, the cap being fixed on the distal end of the endoscope with the proximal opening of the envelope facing the distal end of the endoscope,
a penetration step into said organism to the interior of the cavity through an orifice in this cavity,
a step in which the foreign body is gripped by gripping means of the endoscope,
a step in which the foreign body is surrounded by the envelope of the cap,
a step in which the endoscope is withdrawn from the organism.

This use enables extraction of a perforating and/or cutting foreign body from an organism without injuring the tissues, particularly the edges of the orifice of the cavity and the edges of a passage leading to this cavity.

The gripping means may in particular be a clamp, a hook or a gripping arm.

Use according to embodiments of the invention can optionally include one or several of the following characteristics:
the cap can have a folded configuration and an unfolded configuration, said penetration step being done with the cap in its folded position, the use comprising a step to unfold the envelope from its folded configuration to its unfolded configuration, the unfolding step being done before or during the withdrawal step, before the foreign body has started to come out of the cavity;
according to embodiments of the invention, the use may include features as follows:
in its folded configuration, the envelope of the cap is turned back around the attachment means and is brought up along the endoscope from the distal end of the endoscope,
in its unfolded configuration, the envelope is unwound in front of the end of the endoscope,
the envelope changes from its folded configuration to its unfolded configuration by unwinding the envelope,
the folding step can be done after the step in which the foreign body is gripped;
the step in which the foreign body is surrounded can be done by the unfolding step, the envelope passing from its folded configuration to its unfolded configuration unwinding around the foreign body;
the unfolding step of the cap envelope can be done during the withdrawal step, the distal opening of the envelope bearing on the edges of the orifice of the cavity, such that continuation of withdrawal causes unwinding of the envelope;
according to embodiments of the invention, the use may present features as follows:
in its folded configuration, the cap envelope is in front of the distal end of the endoscope and is in compacted form, the distal opening and the proximal opening facing each other and being open,
in its unfolded configuration, the envelope is wider and tapers outwards from the proximal opening to the distal opening,
the envelope changes from its folded configuration to its unfolded configuration as the envelope unfolds;
the unfolding step can be done before the step to grip the foreign body;
the wall can be elastic such that in the folded configuration, a stress is applied to the wall generating a return force tending to bring the wall from its folded configuration to its unfolded configuration, the envelope being kept under this stress by one or more retaining elements, the unfolding step comprising a sub-step to actuate this or these retaining elements by a actuator of the endoscope so as to eliminate the need to maintain a stress in the envelope, such that the envelope changes to the unfolded position;
the actuator may include a wire or a cable passing through an endoscope operator channel and connected at one end to the retaining element(s) and at the other end to a user interface used to actuate the wire or the cable, the retaining element(s) being actuated by actuation of the wire or the cable;
use may include a step to fix the cap onto the distal end of the endoscope such that this distal end, the proximal opening and the distal opening are all in line;
the attachment step may include insertion of a cap retention sleeve on the distal end of the endoscope;
the attachment step may include placement of the distal end of the endoscope into a cap retention sleeve and placement of an elastic around the retention sleeve so as to hold it on the endoscope;
the attachment step can include:
placement of the cap wall around the distal end of the endoscope,
adjustment of the cap along the endoscope,
placement of an elastic on the part of the cap surrounding this distal end, so as to hold the cap on the endoscope, the elastic defining a narrowed area separating the retention sleeve on one side from a housing in front of the distal end on the other side.

Other advantages could also be apparent to one skilled in the subject after reading the examples illustrated by the appended figures given for illustration purposes.

### BRIEF DESCRIPTION OF THE FIGURES

FIG. 1 represents a cap according to the basic configuration of an embodiment of the invention.

FIG. 2 represents a cap according to an embodiment of the invention, particularly according to FIG. 1, in a folded configuration by turning over.

FIG. 3 represents a cap according to an embodiment of the invention in a configuration folded by compaction.

FIG. 4 represents a cap according to an embodiment of the invention with an internal stiffener.

FIG. 5 represents an endoscope.

FIG. 6 represents a front view of the distal end of the endoscope in FIG. 5.

FIG. 7 represents use of the endoscope with a cap according to an embodiment of the invention.

FIG. 8 represents another use of the endoscope with a cap according to an embodiment of the invention.

FIGS. 9a and 9b represent a cap according to an embodiment of the invention, provided with a slit and mounted on endoscopes with different diameter distal ends.

FIG. 10 represents a cap according to an embodiment of the invention formed by longitudinal rolling.

### DESCRIPTION OF EXAMPLES OF THE CAP AND ITS USE

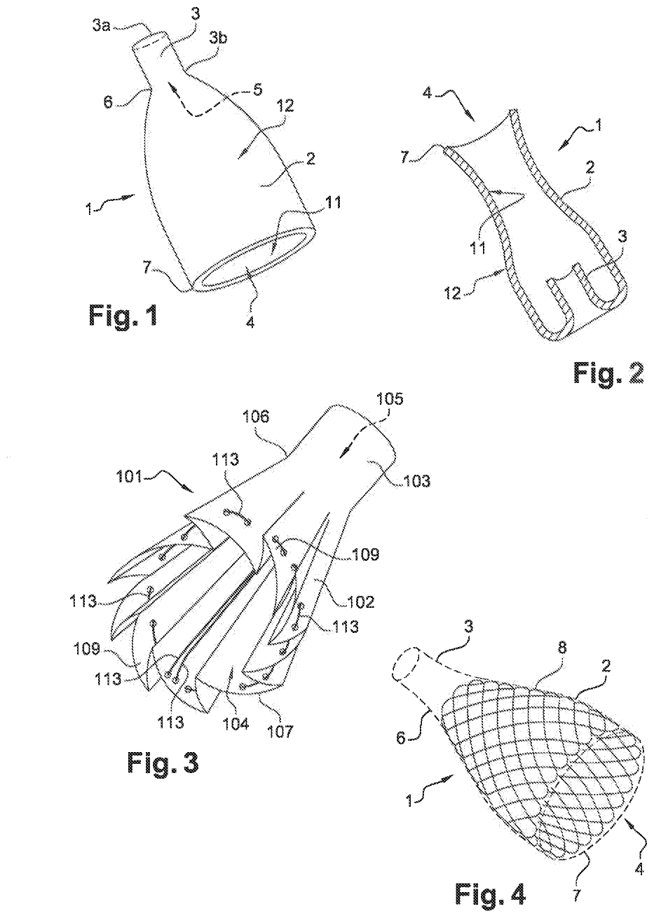

FIG. 1 illustrates a diagram of an example of a cap 1 according to an embodiment of the invention, according to a configuration called the basic configuration.

This comprises a protection envelope 2 formed by a wall folded on itself such that this envelope comprises a proximal opening 5 and a distal opening 4. In other words, the envelope forms a net with an apex 6 comprising the proximal opening 5 and a base 7 comprising the distal opening 4.

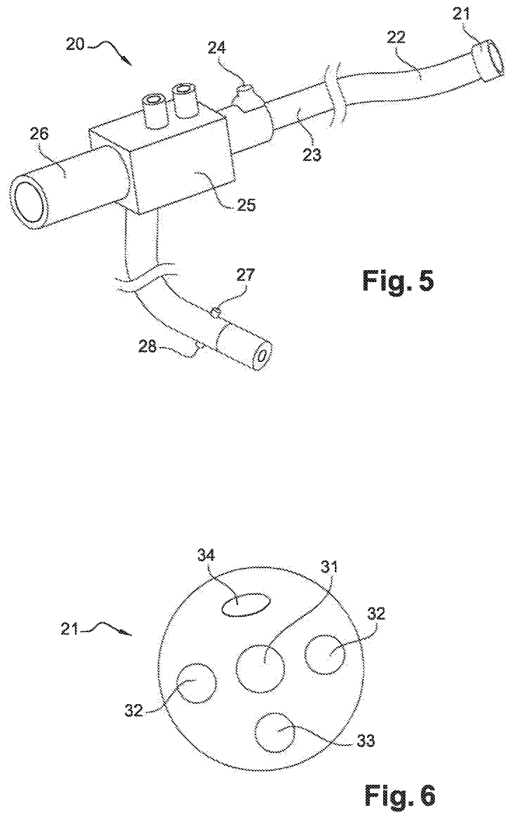

The cap 1 also comprises an attachment means 3 arranged to enable attachment of the cap 1 onto the distal end of an endoscope, as illustrated in FIGS. 5 and 6. The attachment means 3 is arranged so that it is fixed to the distal end 21 of the endoscope 20, so that the proximal opening 5 is facing or is around the distal end 21 of the endoscope 20.

For example, as illustrated, this attachment means is formed by a retention sleeve 3 with a first end 3a and a second end 3b.

In this example, this sleeve 3 will be fitted or slid onto the distal end 21 of the endoscope 20. Thus, the first end 3a of the sleeve 3 is fixed to the distal end 21 of the endoscope 20. The second end 3b of the sleeve opens up in the proximal opening 5.

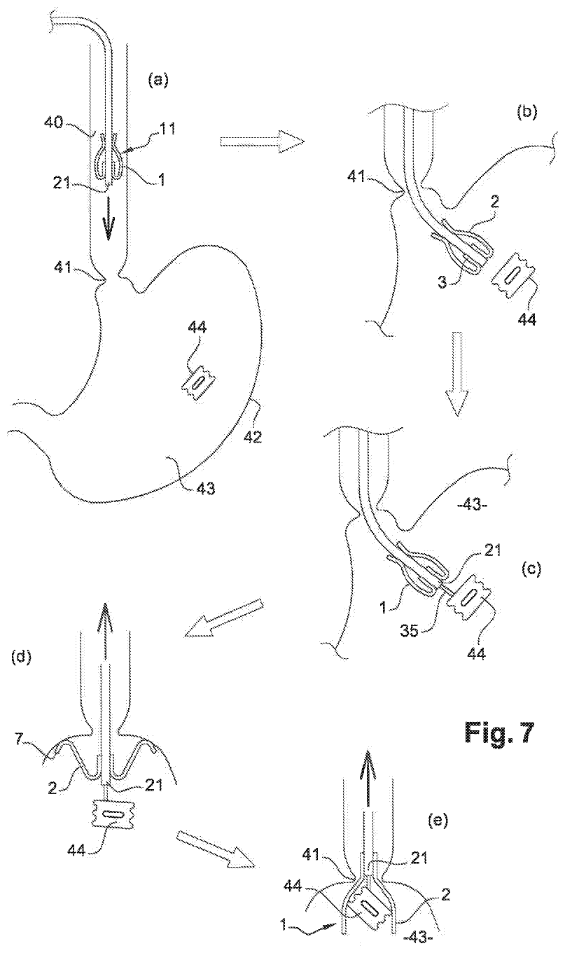

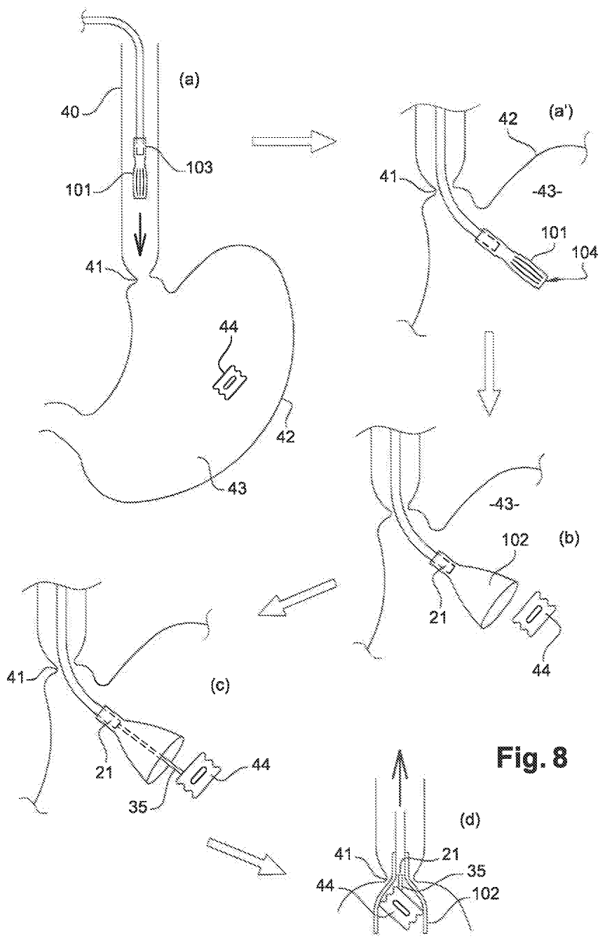

As can be seen on the five steps illustrated in FIGS. 7 and 8, when the sleeve 3 is slid onto the endoscope 20, the distal end 21 is close to the proximal opening 5 of the cap 1. The distal end 21 comprising an optical element 21, such as a lens, connected to an objective 26, so that the endoscopist can see in front of this distal end 21, this arrangement of the cap 1 makes it possible to see beyond the proximal opening 5.

According to the basic configuration of the envelope 2, the distal opening 4 is facing the proximal opening 5 and the envelope has a shape tapered outwards from its proximal opening 5, in this case as far as the distal opening 4. Therefore visibility using the endoscope 20 extends beyond the cap 1.

According to an embodiment of the invention and particularly in this example, the wall of the envelope 2 is made of a structure with sufficient strength to resist perforation and/or incision by a pointed and/or sharp object. Therefore this makes it possible to advance and search for a foreign body in the cavity of an organism, to grip it and to enclose it in the envelope 2 before withdrawing it from the cavity, without injuring the tissues of this cavity or the tissues between this cavity and an orifice of this organism. These uses will be described in detail below.

In this case, the envelope 2 is in the shape of a bell and therefore can completely cover a foreign body inside a housing defined by this envelope 2 in the basic configuration. The proximal opening 5 is arranged at the apex 6 of this bell, and the distal opening 4 is arranged at the base 7 of this bell.

The envelope 2 can thus have dimensions such that its housing contains a volume 4 centimeters (cm) diameter and 5-6 cm long. Thus, the envelope 2, the length of which is more than 5 cm in this case, can encompass object 5 cm long and 4 cm diameter. The diameter of the distal opening 4 can be at least 4 cm. For example, this makes it possible to encompass a needle, a piece of glass or a razor blade. These dimensions are adapted to extraction of a foreign body from the stomach of a human being. For veterinary use, they can be adapted depending on the size of the animal, and particularly the size of the natural passages of this animal.

The sleeve can be approximately tubular and can have a diameter of 9 to 11 millimetres (mm). This size is not limitative and depends on the endoscope with which the cap will be used. For example, a diameter of 9 to 11 mm is adapted to an adult endoscope for which the distal end is generally 11 mm. In this case the sleeve can be fixed by sliding.

In the case of an endoscope for a newborn baby, called a neonatoscope, the diameter can be 5.9 mm. In such a case, the sleeve can be designed with a diameter of 4 to 5.9 mm.

The wall of the envelope 2 can be formed by one only or by more than one polymer. These are polymers with sufficient strength to resist perforation and/or incision by a pointed and/or sharp object.

As an alternative to the above section, as illustrated in FIG. 4, the envelope 2 comprises a stiffener 8.

In this example, the stiffener 8 is arranged on the periphery of the wall of the envelope 2. Therefore this forms a reinforcement around the housing defined by the envelope 2.

The stiffener 8, as illustrated in the example illustrated, can be integrated into almost all, or even all, of the wall of the envelope 2, in this case from the apex 6 to the base 7 of the bell shape of the envelope.

In this case, this stiffener is formed from one or several interlaced wires, thus forming a flexible grid 8.

This grid 8 is made of metal wire(s). This metal grid thus provides good resistance to incision. Therefore the envelope 2 can surround a razor blade without the razor blade passing through it.

The wire(s) forming the grid 8 can be like those used in tubular endoprostheses, also called “stents”, particularly with the same mesh size. Such meshes are described in the document “Van Boeckel, P. G. A., Sijbring, A., Vleggaar F. P. & Siersema, P. D. Systematic review: temporary stent placement for benign rupture or anastomotic leak of the oesophagus. Aliment. Pharmacol. Ther. 33, 1292-1301 (2011).” [8].

The mesh can comprise a mesh size of about 1 mm² along the side.

According to embodiments of the invention, a mesh size can also be chosen such that the sides are less than 1 mm² to reduce the risk of perforation, particularly by needles. In this case, the mesh size can be chosen such that the envelope 2 has sufficient plasticity to deform during the passage through the orifice of the cavity, particularly the cardia in the case in which the cap 1 is used on an endoscope for withdrawal of a foreign body in the stomach.

The material used for this or these wires can be chosen from among a stainless steel, a nitinol, tantalum, alloys of cobalt and chromium and particularly MP35N or MP20N, platinum, titanium.

In particular, these wires can be made of a shape memory metal alloy such as an alloy of nickel and titanium, in quasi-identical or even identical molar proportions. For example, it could be a nickel and titanium alloy, currently called nitinol.

For example, the wire or wires of the grid 8 can be formed from a nickel and titanium alloy containing 50 to 51% of nickel as a molar percentage, namely 55 to 56% as a mass percentage. This alloy is a shape memory material.

In FIG. 4, only the grid 8 is shown in solid lines so that it can be seen. The external surface of the cap 1, and therefore of the envelope 2, is represented by dashed lines.

In this example, the grid 8 is covered by two cover layers on each side of this grid 8. These layers are formed from a cover polymer, in this example identical for each layer.

FIG. 1 can illustrate the cap in FIG. 4. Therefore in this case, the grid 2 cannot be seen on this FIG. 1, because it is covered both on the inside of the housing defined by the envelope 2 by an internal cover layer, and on the outside of this housing by an external cover layer.

In this example, the two cover layers on each side of the grid 8 form a single polymer part comprising a core formed by the grid 8.

The edge of the wall at the bottom 7 of the envelope can be rounded to reduce irritation to the tissues in case of friction. For example, the external cover layer can be folded above the grid, covering the edge of the grid and this forming a hem.

Moreover, as in this example, these layers can be formed in a single piece with the retention sleeve 3. Thus, the wall of the envelope 2 and this sleeve 3 are formed as a single piece, in other words they are made from a single piece of material. For example, the cap 1 can be obtained by insert molding the polymer(s) around the grid 8, the grid being placed in the cavity of the cap mold and the polymer then being injected.

Therefore in this example, the same polymer or mix of polymers necessarily forms the cover layers and the sleeve 3.

However, according to one alternative not shown, the sleeve and the cover layers can be formed from distinct polymers, namely the sleeve polymer(s), the grid internal cover polymer(s), the grid external cover polymer(s). The terms “internal” and “external” relate to the positions of the cover layers in the basic configuration.

Different types of polymers can be used, particular those used in tubular endoprostheses, and particularly those described in a “Van Boeckel. P. G. A., Sijbring, A., Vleggaar, F. P. & Siersema, P. D. Systematic review: temporary stent placement for benign rupture or anastomotic leak of the oesophagus. Aliment. Pharmacol. Ther. 33, 1292-1301 (2011)” [8].

These polymers can be chosen from among silicone, polyurethane, polytetrafluoroethylene (PTFE).

The polymers in the sleeve 3 can be porous to improve adhesion to the end of the endoscope.

The internal and/or external cover polymer(s) can be one or more polymers resistant to perforation. This can further reinforce the envelope 2. This can also reduce the risk that a needle that is being extracted from the organism, passes through the meshes of the grid 8 during withdrawal.

This grid 8 and the polymers, and consequently the wall of the cap 1 have some stiffness and some flexibility.

In the example illustrated in FIGS. 1, 2 and 4, the grid only begins at the apex 7 of the envelope 2 and therefore is exclusively in the envelope 2, the sleeve 3 not containing any grid. Thus, the zone adjacent to the grid 8 at the second end 3b of the sleeve will be more flexible than the wall of the envelope 2 and consequently forms a hinge. This hinge makes it possible to turn the envelope 2 back on itself around the sleeve 3, that is then enclosed by this wall as illustrated in FIG. 2.

During this turning back, the envelope 2 moves from the basic configuration in FIG. 1 into a folded configuration illustrated in FIG. 2. Thus, the inside face 11 of the wall of the housing in the basic configuration becomes the outside in the folded configuration, and the external face 12 of the wall of the housing in the basic configuration becomes the inside in the folded configuration.

The cap 1 can once again change from the folded configuration to the basic configuration by unwinding in the other direction, the basic configuration corresponding to an unfolded configuration.

According to another example illustrated in FIG. 3, the cap 101 in the basic configuration or in the unfolded configuration, is identical to that shown in FIG. 1 but has a folded configuration called the compacted configuration, distinct from the folded configuration in the preceding example.

In this case, the envelope 102 in the compacted configuration has folded portions 109 folded on themselves, along folds extending in the longitudinal direction from the distal opening 104 to the proximal opening 105 of the envelope 102, namely from the apex 106 to the base 107 of the envelope.

This can reduce the taper and therefore the diameter of the envelope 102, to make the passage into the cavity easier.

According to the example illustrated, the folded portions are held folded by a wire or cable 113 arranged around the envelope 102 at the base 107 of the envelope. For example, it may be a polypropylene cable.

In particular, this cable 113 can pass through holes passing through the wall of the envelope 102 and pass from one side of the wall to the other alternatingly like a shoelace.

According to one alternative not shown, these holes do not pass through the wall from side to side, but the cable can simple pass through once, like a stitch into the material of the cover polymer(s) on the inside of the housing defined by the envelope.

This cable 113 is prolonged inside the housing towards the sleeve 103, particularly until it reaches the sleeve. This means that once the cap 101 has been installed on the endoscope, the cable 113 can be activated by an actuator passing through an operator channel 33 of the endoscope 20 to release the necking of the cable 113, the envelope 102 then returning to its basic configuration as shown in FIG. 1 due to the flexibility of the polymer(s).

Alternatively or additionally, the cap 101 may comprise a grid 8 as in the previous example, being made of a shape memory material for which the temperature at the end of the change to the austenitic phase is between 34 and 42 degrees, and particularly between 36 and 40 degrees.

This grid has a memorized shape corresponding to the shape of the basic configuration, for example like the grid 8 in FIG. 4.

Folded portions 109 can be formed by deformation of the grid, therefore torsion of its wires, before the endoscope is inserted into the organism. The shape memory material is then in its martensitic phase.

Once the cap 101 has been introduced into the cavity, the shape memory material is heated and it changes to the austenitic phase. The grid then changes to the memorized shape, and therefore the envelope 102 changes to its basic configuration.

Therefore in such a case, the cap 101 may not have a cable 113. No action by the endoscopist is then necessary to make the cap 101 open.

There are several fabrication methods possible to obtain a cap 1 or 101 with a stiffener.

According to one fabrication method, the material of the grid 8 is metallic and is chosen particularly from among shape memory materials for which the temperature at the end of the change to the austenitic phase is between 34 and 42 degrees, and particularly nitinol as mentioned above.

A method according to an embodiment of the invention comprises:
a step for fabrication and/or conformation of the grid 8 in the form of a handle for which the shape corresponds at least partly to the shape of the basic configuration,
a step to cover both sides of the grid 8 by a cover polymer, particularly by insert molding of a polymer on the grid in a mold with a cavity corresponding to the shape of the sleeve.

During the conformation step of the grid 8, it is possible to make the grid memorize the shape corresponding to the basic configuration. The grid 8 and particularly its wire(s) may be mechanically stressed for this purpose, for example by clamping between a shape and a mating shape or by weaving of the wires. This stress is applied at a sufficiently high temperature so that this shape because the shape memorized by the grid 8, for example 500° C.

The conformation step is then followed by a step to cool the grid 8 below the martensitic phase transformation end temperature of the material of the wire(s) of the grid 8. The grid 8 is then in the martensitic phase in the shape corresponding to the basic configuration.

The cover step is then applied.

If it is required to make a ready-to-use cap 101 in the compacted configuration, as illustrated in FIG. 3, the next step is to compact the grid by forming folded portions 109, while keeping the distal opening 104 facing the proximal opening 105. This compaction step is done below the martensitic phase transformation end temperature of the grid material, The envelope 102 thus keeps its compacted shape, the grid being twisted.

Techniques other than insert molding can be used for the cover step.

In particular, the grid 8 can be impregnated by the polymer(s) by dipping in the material that is then polymerized around the wire(s) of the grid. The sleeve 3, 103 can then be formed separately and then glued to the envelope 2, 102. It is also possible to form the cap entirely with a grid that also extends throughout the sleeve. A simple impregnation is then sufficient.

The cover can also be made by spraying.

In particular, cover techniques disclosed in application US 2007/288088 A1 can be used.

It is also possible to make an internal layer and an external layer made of polymer material(s) with the adapted shape and to glue them or weld them on each side of the grid.

The cap 1, 101 according to embodiments of the invention, particularly according to the examples described, is particularly useful for use of the cap in a method of extracting a foreign body from an internal cavity in a human or animal organism using an endoscope.

An example of an endoscope 20 is illustrated in FIGS. 5 and 6. This example comprises a tube with a flexible section 23 at one end connected to a control module, and a terminal section with controllable curvature 22 at the other end. The latter terminates with a distal end 21 that will be introduced into an organism, particular through a natural channel. The curvature and the orientation of the terminal section 22 are controlled from the control module 25.

The tube comprises optical means that will route optical images in front of the distal end 21, from an optical lens 31 at the distal end 21 as far as an objective 26 located at the control module 25. The operator, namely the endoscopist, can thus see through the objective 26 in front of the endoscope 20.

The distal end 21 also comprises two lighting devices 32 to illuminate in front of the endoscope 20.

The tube comprises an operator channel 33 with an input 24 at the control module 25 and opening up at the distal end 21 through an output from the operator channel. This operator channel 33 can enable passage of a gripping arm, not shown on FIGS. 5 and 6, and actuation means. The arm can then be actuated by the endoscopist and it can be output in front of the distal end 21.

Schematically, a group of inputs 27 and outputs 28 is represented, through which a gas and particularly air can be injected, and water can be injected, and they can also be sucked out by action of the control module 25. The distal end 21 carries a nozzle 34 through which water or air can be delivered, particularly to clean the lens 31.

As described above, according to embodiments of the invention the sleeve 3 can be designed to have a diameter less than or equal to the diameter of the endoscope on which it is fitted. In such a case, for example as illustrated above, the sleeve 3 is slid onto the distal end 21 of the endoscope 20, its elasticity enabling adaptation and retention of the cap 1 on the endoscope.

However when the endoscopes are very different, the attachment can be completed by an elastic. For example, using a cap with a 9 mm diameter sleeve 3, the attachment can be made on an endoscope for adult without using any elastic. This same cap 1 can be used on a 5.9 mm diameter neonatoscope, by sliding the sleeve 3 onto the distal end of the neonatoscope, and then passing an elastic around the sleeve 3.

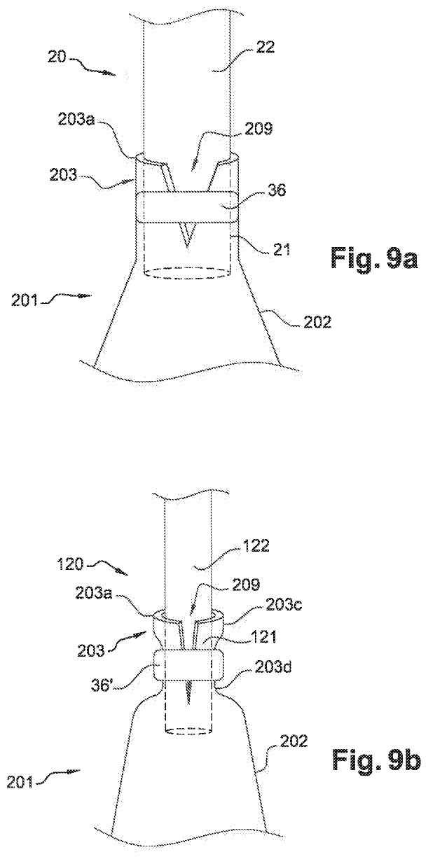

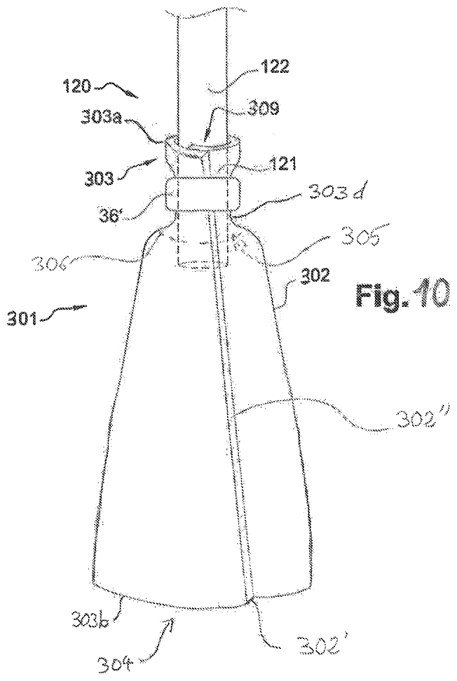

According to embodiments of the invention, and particularly according to the example illustrated in FIGS. 9a and 9b, the sleeve 203 of the cap 201 can comprise a longitudinal slit 209 tapered outwards towards the first end 203a of the sleeve 203.

When the sleeve 203 is mounted on this endoscope 20, as in FIG. 9a, it keeps its approximately cylindrical shape around the distal end 21. An elastic 36 is then passed around the sleeve to hold the sleeve in place.

When the sleeve 203 is mounted on a neonatoscope 120, as in FIG. 9b, the edges of the slit 209 are brought close together and are held in this position by the addition of a smaller or tighter elastic 36′. The elastic 36′ thus separates the sleeve 203 into a downstream part 203d between the envelope 202 and the elastic 36′, and an upstream part at the other end of the elastic 36′. Thus due to this slit, the walls of the sleeve 203, particularly in the upstream part 203c, are brought close to the tube 122 of the neonatoscope 120. This upstream part 203c is thus less tapered than when there is no slit 209. This slit thus improves adaptability of the cap 201 to an endoscope 120 at the distal end 121 with smaller diameter. This also makes it easier to turn the envelope 202 back on itself around the sleeve 203.

In these different alternatives, the wall forming the envelope is continuous around its entire periphery.

According to one alternative, as illustrated on FIG. 10, the wall 302′ forming the envelope 302 is discontinuous and split over its entire length, namely from a proximal end 303a to a distal end 303b. The envelope 302 is obtained by winding the wall 302′ on itself in the direction of the length, so as to close the envelope 302 between the proximal end 303a that will be around the endoscope, in this case the neonatoscope 120, and the distal end 303b, that forms the distal opening 304.

In this case, the cap 301 can be mounted around the endoscope 120 by winding the wall 302′ around the tube 122 of the endoscope, until the wall 302′ at least partially overlaps itself along its length. Thus, a housing has been formed in front of the endoscope 120. In the example illustrated, the zone 309 in which the wall 302′ overlaps itself is limited but it could be more or less large.

One 302″ of the longitudinal slices of the wall 302′ is thus visible at the exterior of the envelope 302.

A fastener such as an elastic 36′, can then be passed around the rolled wall 302′ and the distal end 121 of the endoscope so as to fix the cap 302 to the endoscope 120. The cap 302 thus has the sleeve 303 at one end of the elastic 36′, and the envelope 302 at the other end of this elastic 36′. In other words, the cap 301 is formed by a cape, of which the rolled part and the fastener around the endoscope 120 form the housing between the proximal opening 305 and the distal opening 304 of the envelope 302.

The cape can then comprise a stiffener as described above, particularly a wire mesh (not shown), and particularly wires made of a shape memory material. In this case, the mesh can extend on a downstream portion of the cape, called the reinforced portion. This reinforced portion forms the essential part of this envelope 302.

In the example illustrated, the reinforced portion being between a proximal limit 306 and the distal opening 304 of the envelope 302.

The elastic 36′ can be fixed at a distance from this proximal limit 306 so as to leave a downstream portion 303d of the sleeve 303 just between the elastic 36′ and this proximal limit 306. Since this downstream portion 303d of the sleeve 303 does not have a stiffener, in this case a mesh, it forms a hinge of the envelope so that it can be turned back on itself and above the sleeve 303, before insertion of the endoscope 120 into the patient.

This cape can be made using the fabrication method described above, except that the stiffener manufacturing and/or conformation step is done differently. In this case, the stiffener and particularly a grid is made in the form of a cape and not a handle, for example a trapezoidal strip.

FIG. 7 illustrates an example of the use of the cap 1 in FIGS. 1, 2 and 4 in a method of extraction of a razor blade 44, ingested deliberately and housed inside the cavity 43 defined by the stomach 42 of a human being.

Firstly, the sleeve 3 is slid on the distal end 21 of the endoscope 20, and possibly adjusted in depth. The sleeve may include a rolled edge, not shown, that fits behind the distal end 21 to prevent the risk of loss of the cap 1 in the stomach 42. The attachment can be completed by an elastic.

The envelope 2 is then turned backwards, above and around the sleeve 3.

The endoscopist then implements a penetration step (a) into the organism, through the person's mouth. The endoscope tube is then introduced into the stomach by passing it through the oesophagus 40.

Once the envelope 2 has been turned back, its internal surface 11 is on the outside and it can rub against the walls of the oesophagus 40 or the cardia 41 before entry into the stomach 42. However, the internal cover layer can protect these walls from any irritation, particularly by the grid 8.

The endoscopist then performs a step (b) to search for the razor blade 44. Since the envelope 2 has been turned back, it leaves a very wide field of vision.

Once the blade 44 has been found, the endoscopist performs a step (c) to grip this blade 44 by the gripping arm 35 of the endoscope 20, that comes out of the operator channel 33.

The arm 35 is then retracted and the tube of the endoscope is gradually withdrawn. At a given moment, the distal end 21 moves towards the cardia 41 and the base 7 of the envelope comes into contact with the edges of the cardia 41. The force applied by the withdrawal movement of the distal end 421 from the stomach 42 pushes this base 7 forwards, and causes progressive turning back of the envelope 2. As withdrawal from the cavity 43 takes place, the envelope 2 unrolls and thus surrounds the razor blade 44, before the envelope leaves the stomach cavity 43. Therefore the step (d) to surround the blade takes place during the passage through the cardia 41.

It is then possible to begin the step (e) to withdraw the tube of the endoscope 20 from the organism. As can be seen, the envelope 2 completely surrounds the razor blade 44 and enables a passage through the cardia 41 without injuring the cardia. It will also protect the wall of the oesophagus 40.

Due to its structure, the razor blade 44 will not pass through the envelope 2, despite the friction.

FIG. 8 illustrates an example use of the cap 101 in FIG. 3 in a method of extraction of a razor blade 44, ingested deliberately and housed inside the cavity 43 defined by the stomach 42 of a human being.

Firstly, the sleeve 103 is slid on the distal end 21 of the endoscope 20, and possibly adjusted in depth in the same way as for use in FIG. 7.

The envelope is then folded back by a compaction step, unless the cap 101 has already been compacted before delivery. This compaction step is identical to the step described above with reference to FIG. 3.

The endoscope then uses a penetration step (e) into the organism as far as the stomach cavity 43, through the patient's mouth and then the oesophagus 40, as in the previous example.

In this case the cap 101 comprises a grid made of a shape memory material as described above.

Due to its compacted shape, the cap 101 does not rub very hard on the walls of the organism. Furthermore, the external polymer cover layer can protect the tissues from the grid of the cap.

Like the longitudinal folds, the distal opening 104 remains in front of the proximal opening 105 and enables first guidance.

Once inside the stomach 42, namely in step (a′) in FIG. 8, heat triggers unfolding and expansion of the envelope 102. The bell shape of the envelope 102 provides a good angle of vision through the distal end 21 and the endoscopist can perform a step (b) to search for the razor blade 44.

Once the blade 44 has been found, the endoscopist performs a step (c) to grip this blade 44 by the gripping arm 35 of the endoscope 20, that comes out of the operator channel 33.

The arm 35 is then retracted, bringing the razor blade 44 inside the envelope 102.

It is then possible to begin the step (d) to withdraw the tube of the endoscope from the organism. Also in this example, the envelope 102 completely encloses the razor blade 44 and enables passage through the cardia 41 and the oesophagus 40 without injuring them.

Although these examples have been described in the framework of extracting a foreign body from the stomach of a human being, it could be applied for veterinary use, for example in the case of glass pieces contained in food ingested by the animal.

The foreign body could also be extracted from a cavity other than the stomach. The initial penetration may not have been along a natural path, it could have been following an incision to access the cavity, to avoid causing further injury to the incised tissues.

Another advantage of the cap is that it is easy to produce, particularly making use of materials used in the medical field. Therefore it can be produced at low cost and can thus be more easily used in disposable applications.

### LIST OF REFERENCES

[1] Hesham A-Kader, H. Foreign body ingestion: children like to put objects in their mouth. World J. Pediatr. WJP 6, 301-310 (2010).
[2] Olives, J.-P., Bellaïche, M. & Michaud, L. [Digestive foreign bodies in children]. Arch. Pédiatrie Organe Off. Société Fr. Pédiatrie 16, 962-964 (2009).
[3] Chauvin, A., Viala, J., Marteau, P., Flermann, P. & Dray, X. Management and endoscopy techniques for digestive foreign body and food bolus impaction. Dig. Liver Dis. Off. J. Ital. Soc. Gastroenterol. Ital. Assoc. Study Liver 45, 529-542 (2013).
[4] Goh, B. K. P. et al. Perforation of the Gastrointestinal Tract Secondary to Ingestion of Foreign Bodies. World J. Surg. 30, 372-377 (2006).
[5] Ikenberry, S. O. et al. Management of ingested foreign bodies and food impactions. Gastrointest. Endosc. 73, 1085-1091 (2011).
[6] Velitchkov, N. G., Grigorov, G. I., Losanoff, J. E. & Kjossev, K. T. Ingested foreign bodies of the gastrointestinal tract: retrospective analysis of 542 cases. World J. Surg. 20, 1001-1005 (1996).
[7] Michaud, L., Bellaïche, M. & Olives, J.-P. Ingestion de corps étrangers chez I'enfant. Recommandations du Groupe francophone d'hépatologie, gastroentérologie et nutrition pédiatriques. Arch. Pédiatrie 16, 54-61 (2009).
[8] Van Boeckel, P. G. A., Sijbring, A., Vleggaar, F. P. & Siersema, P. D. Systematic review: temporary stent placement for benign rupture or anastomotic leak of the oesophagus. Aliment. Pharmacol. Ther. 33, 1292-1301 (2011).

1. Endoscopic cap comprising:
a protection envelope formed by a wall folded on itself such that said envelope comprises a proximal opening and a distal opening facing the proximal opening, and the wall having a shape tapered outwards from its proximal opening to its distal opening, in a basic configuration,
attachment means arranged to enable attachment of the cap onto the distal end of an endoscope with said proximal opening facing or around the distal end of the endoscope,
characterized in that the wall of said envelope is made of a structure with sufficient strength to resist perforation and/or incision by a pointed and/or cutting object.

2. Cap according to claim 1, wherein said envelope forms a bell, the proximal opening being arranged at the apex of this bell, and the distal opening being arranged at the base of this bell.

3. Cap according to claim 1, wherein in its basic configuration, said envelope defines a housing and the wall of said envelope includes a stiffener arranged to surround this housing.

4. Cap according to claim 3, wherein the stiffener is composed of one or several wires.

5. Cap according to claim 4, wherein the stiffener can be composed of one or more wires made from a shape memory material.

6. Cap according to claim 4, wherein the wire(s) form(s) a mesh.

7. Cap according to claim 4, wherein said envelope includes at least one layer composed of at least one external cover polymer covering the stiffener at the exterior of said housing.

8. Cap according to claim 7, wherein said envelope includes at least one other layer composed of at least one internal cover polymer covering the stiffener at the interior of said housing.

9. Cap according to claim 1, wherein the wall of said envelope (2; 102; 202) is formed from one polymer only or from several polymers.

10. Cap according to claim 1, wherein the attachment means comprise a retention sleeve intended to be fixed on the distal end of the endoscope, the retention sleeve having a second end fixed on said envelope such that the sleeve opens up in said proximal opening.

11. Cap according to claim 10, wherein the sleeve and the wall of said envelope are a single piece made of the same material.

12. Cap according to claim 10, wherein the sleeve is composed of a material made of one or more polymers, called sleeve polymers.

13. Cap according to claim 12, wherein the material forming the sleeve is elastic and/or porous.

14. Cap according to claim 10, wherein the sleeve has an internal surface comprising at least one recess and/or at least one relief.

15. Cap cording to claim 1, wherein the wall is flexible so that the envelope can be put into a folded configuration, in which the envelope is folded back on itself around the attachment means, and to change from this configuration to an unfolded configuration, corresponding to the basic configuration and in which said envelope is turned back in the other direction such that it is positioned adjacent to the attachment means.

16. Cap according to claim 15 wherein the envelope, in its basic configuration, defines a housing and the wall of the envelope includes a stiffener arranged to surround the housing and, wherein the stiffener only begins at the apex of the envelope and remains exclusively in the envelope, the attachment means not having a stiffener, the zone adjacent to the stiffener thus being more flexible than the wall of the envelope and thus forming a hinge around which the envelope can be turned back on itself in its folded configuration.

17. Cap according to claim 1, wherein the envelope is arranged so as to pass, by deformation, from a compacted configuration to an expanded configuration corresponding to the basic configuration, in its compacted configuration the envelope having a smaller taper than in the basic configuration or not having a taper at all, while keeping the distal opening (104) facing the proximal opening.

18. Cap according to any claim 1, wherein the wall forming the envelope is discontinuous and split over its entire length, from a proximal end to a distal end, such that the envelope can be obtained by rolling the wall on itself along the direction of its length, so as to close the envelope between the proximal end (303a) intended to be around the endoscope, and the distal end that thus forms the edges of the distal opening.

19. Method of fabrication of a cap for an endoscope, said cap complying with any one of claim 1, said cap comprising an envelope comprising a proximal opening and a distal opening facing the proximal opening and having a shape of a basic configuration, this shape tapering outwards from its proximal opening towards its distal opening, said fabrication method comprising:
a step for fabrication and/or conformation of a stiffener in the form of a handle for which the shape corresponds at least partly to the shape of the basic configuration, namely in the form of a cape,
a step to cover the stiffener by a layer composed of at least one covering polymer on at least one of the stiffener faces,
the stiffener material and/or the covering polymer(s) being resistant to perforation and/or incision by a pointed and/or cutting object, said manufacturing method.

20. Use of a cap according to claim 1, in a method for extraction of a foreign body from an internal cavity (43) in a human or animal organism, comprising:
a step to pick up an endoscope, fitted with a cap, the cap being fixed on the distal end of the endoscope with the proximal opening of said envelope facing the distal end of the endoscope,
a penetration step (a) into said organism to the interior of said cavity through an orifice in this cavity,
a step (c) in which the foreign body is gripped by a gripping means of the endoscope,
a step (d) in which the foreign body is surrounded by the envelope of the cap,
a step (e) in which the endoscope is withdrawn from the organism.
